(19) United States
(12) Patent Application Publication
(10) Pub. No.: US 2023/0018091 A1
(43) Pub. Date: Jan. 19, 2023

(54) MEDICAL ELECTRODE AND SYSTEM THEREOF

(71) Applicant: INNACCEL TECHNOLOGIES PRIVATE LIMITED, Sampangiramanagar Bangalore Karnataka (IN)

(72) Inventors: Vibhav JOSHI, Sampangiramanagar Bangalore (IN); Sumedh KAULGUD, Sampangiramanagar Bangalore (IN); Aditya MATHUR, Sampangiramanagar Bangalore (IN); Adaikkammai S, Sampangiramanagar Bangalore (IN)

(21) Appl. No.: 17/947,050

(22) Filed: Sep. 16, 2022

Related U.S. Application Data
(63) Continuation of application No. PCT/IN2021/050306, filed on Mar. 24, 2021.

(30) Foreign Application Priority Data
Mar. 25, 2020 (IN) 201941038749

(51) Int. Cl. A61B 5/266; A61B 5/288; A61B 5/265

(52) U.S. Cl. CPC A61B 5/266; A61B 5/288; A61B 5/265; A61B 2562/164; A61B 2562/166

(57) ABSTRACT

The instant invention is a medical electrode comprising: an electrolyte; an absorbent material being in contact with the electrolyte, said absorbent material comprising a plurality of strands such that each strand is infused with the electrolyte; a pressing means being in contact with the absorbent material, said pressing means comprising a plurality of protrusions to push the strands of the absorbent material through the stratum corneum of a target skin area on a patient; and an electrically conductive means located adjacent to the pressing means, said electrically conductive means being in contact with the absorbent material. The instant medical electrode system achieves reduction of skin impedance at the site where the electrode is placed and generates low baseline noise, enabling accurate measurement of small or weak signals (FIG. 1).

### CLAIM OF PRIORITY

This application claims priority as a continuation of International Patent Application No. PCT/IN2021/050306, filed Mar. 24, 2021, now International Patent Publication No. WO 2021/191930, which claims the benefit of Indian Patent Application No. 201941038749, filed Mar. 25, 2020, each of which is herein incorporated by reference in its entirety.

### FIELD

The present invention relates to a medical electrode and system thereof. The instant medical electrode achieves reduction of skin impedance at the site where the electrode is placed, without exfoliating the skin, and generates low baseline noise, enabling accurate measurement of small or weak signals including but not limited to fetal electrocardiogram (ECG).

### BACKGROUND

Medical Electrodes are used to detect electrical potential on the skin surface towards various health diagnoses. Medical electrodes that can effectively sense and detect small or weak electrical signals remain a need in the market. In fetal monitoring techniques, fetal heart rate monitoring is performed using fetal ECG. Surface electrodes with wet gel or solid get are used to pick up this signal. However, the acquisition of fetal ECG signal from the surface of the mother's abdomen is challenging as fetal ECG is of the amplitude of 5-20 microVolts. Further, this signal is hidden in a complex of maternal ECG, signals contributed by uterine activity due to maternal contractions, and ambient noise. Picking up this small signal is made more challenging by the high electrical impedance of the stratum corneum—the topmost layer of skin. The stratum corneum damps down fetal ECG and leads to noise at the electrode skin interface. When measuring fetal ECG, the electrode is placed on the patient's belly. To reduce skin impedance at the site where the electrode is placed, a mildly abrasive paper tape is used to exfoliate the stratum corneum. Prior art electrodes like those used by the Monica electronic fetal monitor have electrodes with fixed gel area, available over a range of sizes, wherein each size requires a minimum number of exfoliation strokes with an abrasive material, for reducing skin associated impedance and noise signals to acceptable levels. However, this approach is skill dependent and often requires multiple attempts which cause discomfort to the patient and leaves scars. Thus, there is a need for an electrode that is able to generate low base line noise and reduce impedance caused by the skin, to detect small or weak signals, without requiring exfoliation of skin at the site where the electrode contacts the skin.

Another prior art electrode by Aspect Medical systems uses tines to part the topmost layer of skin and keep it parted. A gel electrolyte held in a sponge is depressed when the tines are pressed upon and this gel electrolyte seeps into the channels created by the tines. However, this approach is limited as the action of pressing on the tines and its connected electrical pad leads to uneven distribution of the gel and air pockets form between the skin-electrode interface and the electrical pad. The sponge is composed of multiple cells that hold the electrolyte gel. However, some of the sponge cells contain air. When the sponge is depressed, the air gets squeezed out along with the gel, creating air bubbles in the electrode system leading to uneven electrolyte distribution and high base line noise. Thus, there is a need for an electrode that evenly distributes the electrolyte, on application of pressure, without the generation of air bubbles.

Further, this system is particularly ineffective when the electrode is placed on an area of skin that lacks bony structures below e.g., abdominal skin. Pressing on the tines through the sponge into such skin leads to poor parting of the skin. Additionally, the air pockets lead to high electrical noise that masks small or weak signals including but not limited to fetal ECG. Thus, the afore system would be particularly ineffective for fetal monitoring as the electrode would be placed on maternal abdominal skin. Also, as the mother is not under anesthesia, multiple pressing attempts to get the electrode to part the skin, to measure the small fetal signals, make it uncomfortable for her. Hence, there is a need for an electrode that is able to measure signals on any part of the body, particularly on parts that lack a bony structure beneath the skin.

There is also a need for a low-cost electrode as the commercially available electrodes are expensive.

Thus, there is a need for a low-cost medical electrode which effectively reduces skin impedance at the electrode skin interface, has low baseline noise, permits even distribution of electrolyte without air bubbles, can be placed on any part of the body, and avoids exfoliation of skin prior to placement of electrodes reducing patient discomfort at the time of electrode placement.

### SUMMARY OF THE DISCLOSURE

In one embodiment the instant invention is a medical electrode comprising: an electrolyte; an absorbent material being in contact with the electrolyte, said absorbent material comprising a plurality of strands such that each strand is infused with the electrolyte; a pressing means being in contact with the absorbent material, said pressing means comprising a plurality of protrusions to push the strands of the absorbent material through the stratum corneum of a target skin area on a patient; an electrically conductive means located adjacent to the pressing means, said electrically conductive means being in contact with the absorbent material, wherein each electrolyte infused strand of the absorbent material functions as a conductive channel from the site of ingress in the stratum corneum to the electrically conductive means; and a support means for supporting the electrode and to hold the electrode in contact with a target skin area of a patient.

In one embodiment the invention is a medical electrode system comprising the at least one electrode, said at least one electrode being connected to a flexible base that removably engages with a measuring device.

In one embodiment the invention is a method of using the medical electrode or the medical electrode system comprising placing the electrode directly on the target skin area of a patient; pressing the electrode into the skin to push the electrolyte infused strands of the absorbent material through the stratum corneum; and displaying biopotential signal values detected by the electrode on a measuring device.

### BRIEF DESCRIPTION OF THE DRAWINGS

Reference will be made to embodiments of the invention, examples of which may be illustrated in accompanying figures. These figures are intended to be illustrative, not limiting. Although the invention is generally described in context of these embodiments, it should be understood that it is not intended to limit the scope of the invention to these particular embodiments.

FIG. 1A: An embodiment of the medical electrode of the instant invention

FIG. 1B, 1C, 1D: Depict the medical electrode of FIG. 1A being pressed into a target skin area

FIG. 1E: Depicts an embodiment being a multi-electrode system.

### DETAILED DESCRIPTION

The instant invention intends to address the afore stated technical and economic disadvantages by providing a medical electrode comprising: an electrolyte; an absorbent material being in contact with the electrolyte, said absorbent material comprising a plurality of strands such that each strand is infused with the electrolyte; a pressing means being in contact with the absorbent material, said pressing means comprising a plurality of protrusions to push the strands of the absorbent material through the stratum corneum of a target skin area on a patient; an electrically conductive means located adjacent to the pressing means, said electrically conductive means being in contact with the absorbent material, wherein each electrolyte infused strand of the absorbent material functions as a conductive channel from the site of ingress in the stratum corneum to the electrically conductive means; and a support means for supporting the electrode and to hold the electrode in contact with a target skin area of a patient.

The instant electrode creates micro perforations in the top most dead skin layer (the stratum corneum) of the skin when pressure is applied via the pressing means. The pressing action causes the strands of the gauze to be pushed into these perforations. Further, minimal pressure application is required as the gauze is thin and easily penetrates through the skin and sticks to the ingress sites. The strands being pushed into these perforations also eliminates the need for the preparatory exfoliation step at the site at which the electrode is placed. The gel infused strands make contact with the more conductive layers of the skin underneath the stratum corneum. The gel infused strands form electrically conductive channels between the sites of ingress of the stratum corneum and the electrically conductive means of the instant electrode. Since the gel is infused in the gauze strand, it does not spread unevenly or have air bubbles which disrupt the signal transmission. In this manner, the instant electrode allows for the reduction of skin impedance, without exfoliating the skin, and generates low baseline noise leading to the accurate acquisition of weak signals including but not limited to fetal ECG.

The electrolyte gel which is in contact with the absorbent material is the electrolytic medium which allows ionic exchange between the skin surface and the electrically conductive means. The electrolyte is the medium through which biopotential measurement occurs. In one embodiment the electrolyte gel is liquid, slightly tacky or sticky as it is alginate based, highly fluid and a skin-friendly composition. The liquid nature and stickiness ensure complete absorption of the electrolyte by the gauze and enables even spreading when pressure is applied, avoiding air bubble formation.

In one embodiment of the medical electrode the absorbent material comprises the plurality of strands in a network, preferably in the gauze structure. In a preferred embodiment of the medical electrode the absorbent material is of cotton fibers. In the prior art sponge is the absorbent material used in the medical electrodes, said sponge is composed of multiple cells which hold the electrolyte gel. When the sponge is pressed, the gel in the cells is squeezed out. However, electrolyte distribution is often uneven as some of the sponge cells have trapped air and air bubbles are introduced into the system. These air bubbles are to be avoided as they cause high base line noise. Instead, the instant invention uses cotton fibers arranged in the gauze structure. The electrolyte gel gets absorbed into the individual strands of the gauze and does not get squeezed out when the strands are pressed on, the cotton strand itself becomes a conductive channel.

In one embodiment of the medical electrode the pressing means is a plastic micro-bristle structure in contact with a plurality of strands of the absorbent material. In another embodiment the pressing means has a further means on a top surface of the electrode to aid application of pressure on the electrode, said top surface being the surface facing away from the target skin area. The said further means being an external mechanical or electromechanical means of applying pressure onto the electrode. Said further means applies a range of pressure for a specific amount of time due to physical, electrical or chemical characteristics. In one embodiment the further means is a bubble on top of the electrode with a standard amount of air inside the bubble. A specific amount/range of pressure is required to be applied to burst it. Till the bubble is burst, the pressure built up inside is applied evenly across the bottom surface of the bubble, which is aligned with the pressing means on the top surface of the medical electrode, said surface being the one facing away from the skin. In another embodiment the further means is a plastic disk designed to crack once a specific amount of pressure is reached, up to that point it distributes the pressure evenly.

In one embodiment of the medical electrode the electrically conductive means is located adjacent to the pressing means. The term adjacent in this context includes the electrically conductive means being placed near, next to, by the side of, on top of, or offset to the pressing means. In one embodiment of the medical electrode the electrically conductive means is a plastic substrate coated with a conductive metallic layer on the surface in contact with the absorbent material, said surface facing the target skin area. In one embodiment of the medical electrode the conductive metallic layer is of silver/silver chloride. In one embodiment of the medical electrode the plastic substrate is electrically attached to a flexible printed circuit board. In one embodiment the electrically conductive means has a stainless-steel component on the top surface which interfaces/locks into standard electrocardiogram cable connectors to form a connection with any measuring device including but not limited to a fetal or adult ECG device, neural signal measurement device (e.g., EEG) etc. The stainless-steel component includes but is not limited to a stud, a wire, crocodile clip connector, banana stud connector, or a universal snap and tab connector.

In one embodiment the medical electrode is connected to a measuring device for measuring and recording biopotential signals detected by the electrode. This electrode can be utilized for measuring any biopotential signals including but not limited to fetal and adult ECG measurement, neural signal measurement etc. The instant invention is particularly designed to detect and measure small or weak signals through the skin as it reduces skin impedance and generates low baseline noise at the site of ingress of the instant electrode, without requiring exfoliation of the skin at the site of electrode contact.

In one embodiment the support means is for supporting the electrode and to hold the electrode in contact with a target skin area of a patient. In one embodiment the support means comprises two tapes, a backing tape on which is assembled the electrode components and a foam-based pressure sensitive adhesive for adhering the assembled electrode to the skin of the patient. In another embodiment the support means further consists of a covering being a sheet, a film, or a membrane with liquid resistant characteristics on the top surface of the electrode, said top surface being away from the skin of the patient.

In one embodiment the medical electrode comprises a medical electrode system comprising at least one electrode, said at least one electrode being connected to a flexible base that removably engages with a measuring device. The at least one electrode system comprises more than one electrode being connected to a flexible base to create a multi-electrode system, said multi-electrode system being connected to a measuring device.

In one embodiment it is a method of using the medical electrode or the medical electrode system comprising more than one electrode, said method comprising placing the electrode directly on the target skin area of a patient; pressing the electrode into the skin to push the electrolyte infused strands of the absorbent material through the stratum corneum; and displaying biopotential signal values detected by the electrode on a measuring device.

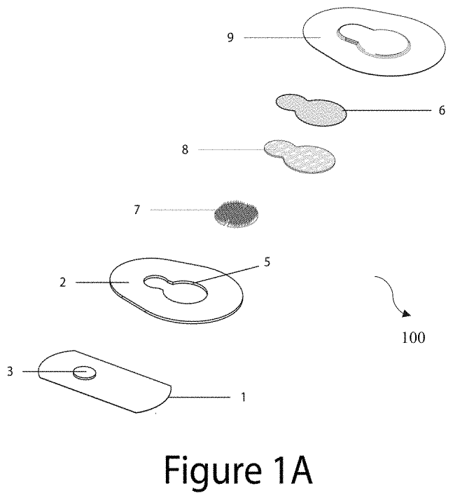

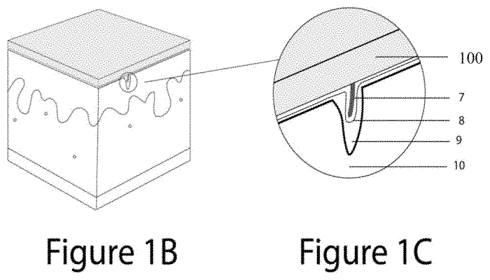

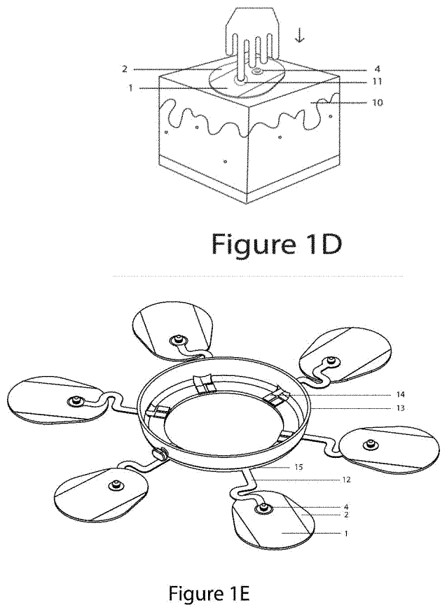

FIG. 1A shows an embodiment of the medical electrode of the instant invention. The figure depicts the medical electrode (100) in its disassembled form, comprising a single layer or multiple layers of soft cotton in gauze or gauze-like structure (8) infused with electrolyte (6), a pressing means (7) in contact with the cotton gauze (8) (in the assembled form) for pressing the strands of gauze into the skin at multiple locations, and an electrically conductive means (3). The pressing means (7) in this embodiment comprises a plastic micro-bristle structure with vertical plastic bristles. The strands of the gauze (8) form electrical channels between the site of the ingress (9) into the stratum corneum (10) and the electrically conductive means (3) (FIG. 1C). The gauze (8), electrically conductive means (3) and pressing means (7) are assembled on a backing tape (1) enclosed in a foam-based pressure-sensitive adhesive (2). The cavity (5) in the pressure sensitive adhesive is shaped so as to concentrate the gauze strands at the juncture with the electrically conductive means (3). In this embodiment the electrically conductive means (3) is offset from the pressing means (7), so pressure is applied directly over the gauze (8).

Assembly of the electrode involves placing the electrically conductive means (3) on the adhesive side of the backing tape (1), said adhesive side facing towards the patient's skin. The pressing means (7) with the micro bristles is stuck offset from the electrically conductive means (3) onto this adhesive side with the bristles pointing towards the skin side. This structure is now assembled into a cavity (5) in a foam-based pressure-sensitive adhesive (2) so that the adhesive side of the pressure-sensitive adhesive is facing towards the skin. The cavity is closed by the backing tape so the system is closed from the top side, the surface not facing the skin. The gauze (8) is placed in cavity (5) to occupy the remaining volume of the cavity and the electrolyte gel (6) is added. The gauze absorbs the electrolyte gel. The gel is the electrolytic medium which allows ionic exchange between the skin surface and the electrically conductive means coated with silver/silver chloride. The electrolyte is the medium through which the biopotential measurement occurs.

FIGS. 1B, 1C and 1D depict the medical electrode (100) of FIG. 1A being pressed into a target skin area. The plastic bristles of the pressing means (7) push the strands of the absorbent material (8) into the stratum corneum (10) at the site of ingress (9). FIG. 1D shows when pressure is applied on the pressing means (arrow depicts the direction of pressure) to push the bristles, hence the electrolyte infused strands of the absorbent material, into the stratum corneum (10). The electrode has a stainless-steel component (4) on the top surface which interfaces/locks into standard electrocardiogram cable connectors to form a connection with any measuring device. The electrode in this embodiment has a further means (11) on the top surface to aid application of pressure on the electrode.

FIG. 1E depicts an embodiment being a multi-electrode system. In this embodiment the multi-electrode patch comprises six electrodes. The electrodes are connected to a flexible base, for removably engaging with a monitoring device for detecting a maternal and/or fetal electrophysiological signal from the electrodes. The flexible base in this embodiment comprises a flexible substrate (12), a plastic unit (13) and a pressure-sensitive adhesive foam ring (15) for attaching the base of the multi-electrode patch to the skin of the patient. The module has a mechanism for removable mechanical engagement with the monitoring device, and an electrical connection unit (14) for making an electrical connection from the electrodes to the readout device. Engaging the patch with the monitoring device comprises both the mechanical module unit and the electrical module unit.

The following experimental example is illustrative of the invention but not limitative of the scope thereof:

### WORKING EXAMPLE 1

The noise characteristics of electrodes of the instant invention versus the prior art electrodes was performed. The instant electrodes comprise an absorbent material being cotton gauze while the prior art electrodes comprise the sponge-based systems. The two electrodes were applied on different locations on the abdomen at a distance of 6 cm from each other. The noise at the sites was measured using 3M solid gel electrodes and was found to be equivalent. These values were used as a base-line of the noise level prior to application of the sponge or gauze electrode.

The study sampled 40 subjects of different ages, gender, and skin types. The results of 5 scans are shown in the table below. The noise detected by the cotton gauze electrode is at least 3-fold lower than the sponge-based electrode, with subject 2 showing a 20-fold reduction in noise.

| | Gauze | Sponge | | |
|---|---|---|---|---|
| | electrode site | electrode site | | |
| | control noise | control noise | Gauze | |
| Subject | (3M solid gel | (3M solid gel | Electrode | Sponge Electrode |
| no | electrodes) | electrodes) | Noise | Noise |
| 1 | 60 microV | 53 microV | 5 microV | 17 microV |
| 2 | 48 microV | 48 microV | 1.2 microV | 22 microV |
| 3 | 85 microV | 83 microV | 3.5 microV | 24 microV |
| 4 | 24 microV | 29 microV | 1.5 microV | 18 microV |
| 5 | 47 microV | 52 microV | 4 microV | 20 microV |

Thus, the instant invention comprises a low-cost medical electrode that effectively reduces skin impedance at the electrode skin interface, has low base line noise signals, avoids exfoliation of skin prior to placement of electrodes, permits even distribution of electrolyte without air bubbles, can be placed on any part of the body, and reduces patient discomfort at the time of electrode placement.

While the present invention has been described with respect to certain embodiments, it will be apparent to those skilled in the art that various changes and modifications may be made without departing from the scope of the invention as defined in the following claims.

What is claimed is:

1. A medical electrode comprising:
an absorbent material in contact with an electrolyte, wherein the absorbent material comprises a plurality of strands such that each strand is infused with the electrolyte;
a press in contact with the absorbent material, the press comprising a plurality of protrusions configured to push the plurality of strands of the absorbent material through a stratum corneum of a target skin area on a patient;
an electrically conductive substrate adjacent to the press, the electrically conductive substrate in electrical contact with the absorbent material, wherein each electrolyte infused strand of the absorbent material is configured to function as a conductive channel from a site of ingress in the stratum corneum to the electrically conductive substrate; and
a support configured to hold the medical electrode in contact with the target skin area.

2. The medical electrode of claim 1, wherein the absorbent material comprises a gauze or gauze structure.

3. The medical electrode of claim 1, wherein the absorbent material comprises cotton fibers.

4. The medical electrode of claim 1, wherein the electrically conductive substrate is a plastic substrate coated with a conductive metallic layer on a surface that is in contact with the absorbent material and is configured to face the target skin area.

5. The medical electrode of claim 4, wherein the conductive metallic layer is silver/silver chloride.

6. The medical electrode of claim 4, wherein the plastic substrate is connected to a flexible printed circuit board.

7. The medical electrode of claim 1, wherein a top surface of the press is configured to distribute applied pressure on the medical electrode.

8. The medical electrode of claim 1, wherein the press comprises a micro-bristle structure.

9. The medical electrode of claim 1, wherein the support comprises a foam material comprising a pressure-sensitive adhesive surrounding the absorbent material.

10. The medical electrode of claim 1, wherein the electrically conductive substrate is in electrical contact with the absorbent material through a layer of electrolyte gel on the electrically conductive substrate.

11. The medical electrode of claim 1, further comprising an electrical contact in electrical communication with the electrically conductive substrate that is configured to couple to an electrical cable.

12. The medical electrode of claim 1, further comprising a flexible base configured to removably engage with a measuring device.

13. The medical electrode of claim 1, wherein the electrically conductive substrate is offset from the press and absorbent material so that pressure applied by the press is applied directly over the absorbent material.

14. A medical electrode comprising:
an absorbent material in contact with a layer of an electrolyte gel, wherein the absorbent material comprises a plurality of strands, wherein each strand of the plurality of strands is infused with the electrolyte gel;
a press in contact with the absorbent material, the press comprising a plurality of protrusions configured to drive the plurality of strands of the absorbent material through a stratum corneum of a target skin area on a patient;
an electrically conductive substrate in electrical contact with the absorbent material through the layer of electrolyte gel, wherein each electrolyte infused strand of the absorbent material is configured to function as a conductive channel from a site of ingress in the stratum corneum to the electrically conductive substrate;
an electrical contact on an outer surface of the medical electrode and in electrical communication with the electrically conductive substrate; and
an adhesive support configured to secure the medical electrode in contact with the target skin area.

15. A method of using a medical electrode, the method comprising:
placing a medical electrode on a target skin area of a patient;
pressing on a press of the medical electrode comprising a plurality of protrusions to drive a plurality of strands of an absorbent material through a stratum corneum of the target skin area, wherein each strand of the plurality of strands is infused with an electrolyte gel;
forming an electrically conductive channel from a site of ingress in the stratum corneum to an electrically conductive substrate of the medical electrode through the plurality of strands; and
detecting a signal from the electrically conductive substrate.

16. The method of claim 15, wherein placing the medical electrode on the target skin area comprises adhesively securing the medical electrode to the target skin area.

17. The method of claim 15, wherein placing the medical electrode on the target skin area comprises placing the medical electrode without exfoliating the target skin area first.

18. The method of claim 15, wherein pressing on the press comprises pressing on a micro-bristle structure.

19. The method of claim 15, wherein pressing on the press to drive the plurality of strands of the absorbent material comprises driving the plurality of strands of cotton fibers through the stratum corneum.

20. The method of claim 15, further comprising displaying the signal detected on a measuring device.
